(19) United States
(12) Patent Application Publication
(10) Pub. No.: US 2024/0118256 A1
(43) Pub. Date: Apr. 11, 2024

(54) OPTICAL METHOD OF EXAMINING THE LIQUID SUBSTANCE OF AN EGG

(71) Applicant: Zvi NEZER, Raanana (IL)

(72) Inventor: Zvi NEZER, Raanana (IL)

(21) Appl. No.: 18/268,982

(22) PCT Filed: Jan. 4, 2022
(86) PCT No.: PCT/IL2022/050013

§ 371 Date: Jun. 22, 2023

(30) Foreign Application Priority Data
Jan. 19, 2021 (IL) 280284

(51) Int. Cl. G01N 33/08

(52) U.S. Cl. CPC G01N 33/085

(57) ABSTRACT

An optical method of examining the liquid content of an egg by illuminating the egg, which results in illumination intensity distribution to appear on the eggshell of the egg, the illumination intensity distribution is dependent on both the structure of the eggshell and the nature of the egg's contents, the method comprising obtaining at various intervals of time images of the eggshell surface with illumination intensity distribution, wherein at each time interval a force acting on the egg is changed in such a way that the force change does not have any observable effect on the eggshell, but causes a change in the liquid contents of the egg, either in the position of the contents as a whole or in the position of particles within the liquid contents, thus changing the illumination intensity distribution appearing on the eggshell, comparing the obtained images of the eggshell and identifying differences in illumination intensity distributions, thus allowing optical examination of egg's liquid content with minimal optical interference from illumination intensity distribution of the eggshell.

### INTRODUCTION

The invention relates to egg examination methods.

In the egg market there is a need to examine the liquid contents of the egg without damaging the eggshell. For instance, it is essential to check whether an egg is fertilized or not; moreover, it is necessary to identify defective contents of the egg that lowers its quality.

One of the common methods for such testing is by illuminating the egg with a powerful flashlight and visually examining the distribution of radiation across the eggshell due to the passage of light through the liquid content of the egg.

The difficulties in such testing are the presence of defects in the eggshell, unevenness of the eggshell and variation in the colors of the eggshell which may obscure the radiation distribution across the eggshell due to the passage of the rays through the liquid content of the egg.

The requested invention is intended to solve some of the difficulties specified above and reduce their impact.

A large number of patents, mainly concerning the subject of testing fertilized eggs, have been published, primarily to identify the embryo within them. The following patents are based on the illumination of the egg and the investigation of the radiation scattered from the eggshell; however, none of these exploits the physical properties of the liquid content of the egg as opposed to the rigidity of the eggshell.

Patent U.S. Pat. No. 3,540,824 relates to the illumination of the egg at wavelengths in the visible range and testing of fetal heartbeat.

Patent EP 2446258A2 relates to illumination of the egg by infrared sources and examination of the fetal heart.

Patent U.S. Pat. No. 8,724,098 relates to the illumination of the egg using wavelengths in the 440-570 nm range and photography of the eggshell to analyze the capillary network of the fetus.

Patent U.S. Pat. No. 9,395,346 relates to the remote electromagnetic sensing of cardiac activity within eggs moving on a conveyor belt.

Patent US2008/0252877A1 relates to photography of the illuminated egg using a wavelength absorbed by the blood and repeating the photograph with illumination by a wavelength that is not absorbed by the blood. The comparison of the two photographs allows identification of the presence of blood in the egg. This patent uses comparison between photographs when the difference between the photographs is at the wavelength at which the photograph is taken.

None of these and other patents refers to or is based on the liquid property of the egg contents and the fact that the optical properties of this medium can be analyzed with the aid of mechanical influences. Thus, the proposed method boasts an inventive innovation and the possibility of practical application.

### LIST OF FIGURES

FIG. 1 is a schematic representation of an experimental setup for examining the liquid content of an egg.

FIG. 2 is a side and a top view of an illuminated egg.

FIG. 3 is a side and a top view of an illuminated egg having centrifugal force applied to it.

FIG. 4 is a side and a top view of an illuminated egg having increased centrifugal force applied to it.

FIG. 5, 6, 7 are schematic representations of another experimental setups for examining the liquid content of an egg.

### DESCRIPTION

General Description

An optical method of examining the liquid content of an egg by illuminating the egg is disclosed. Distribution of the intensity of illumination on the eggshell is dependent on both the structure of the eggshell and the nature of the contents. The eggshell is photographed at various intervals of time, when at each interval the force exerted on the egg is changed in such a way that the force does not have any influence on the hard eggshell, but causes a change in the liquid contents, either in the position of the contents as a whole or in the position of particles within the liquid. Comparison of the images or photographs, in which the participation of the eggshell is minimized, gives an indication of differences in the intensity distribution of light rays as the liquid contents move around inside the egg and enables the analysis of these contents.

The present invention is an optical method of examining the liquid content of an egg by illuminating the egg, which results in illumination intensity distribution to appear on the eggshell of the egg, the illumination intensity distribution is dependent on both the structure of the eggshell and the nature of the egg's contents, the method comprising obtaining at various intervals of time images/photographs of the eggshell surface with illumination intensity distribution, wherein at each time interval a force acting on the egg is changed in such a way that the force change does not have any observable effect on the eggshell, but causes a change in the liquid contents of the egg, either in the position of the contents as a whole or in the position of particles within the liquid contents, thus changing the illumination intensity distribution appearing on the eggshell; comparing the obtained images/photographs of the eggshell and identifying differences in illumination intensity distributions, thus allowing optical examination of egg's liquid content with minimal optical interference from illumination intensity distribution of the eggshell.

If a position of the illuminated egg in one image is close but not identical to a position of the same illuminated egg in another image, the positional discrepancy between the images can be minimized or totally removed by digitally manipulating the images or image data, in order to differentiate between the images.

### DETAILED DESCRIPTION

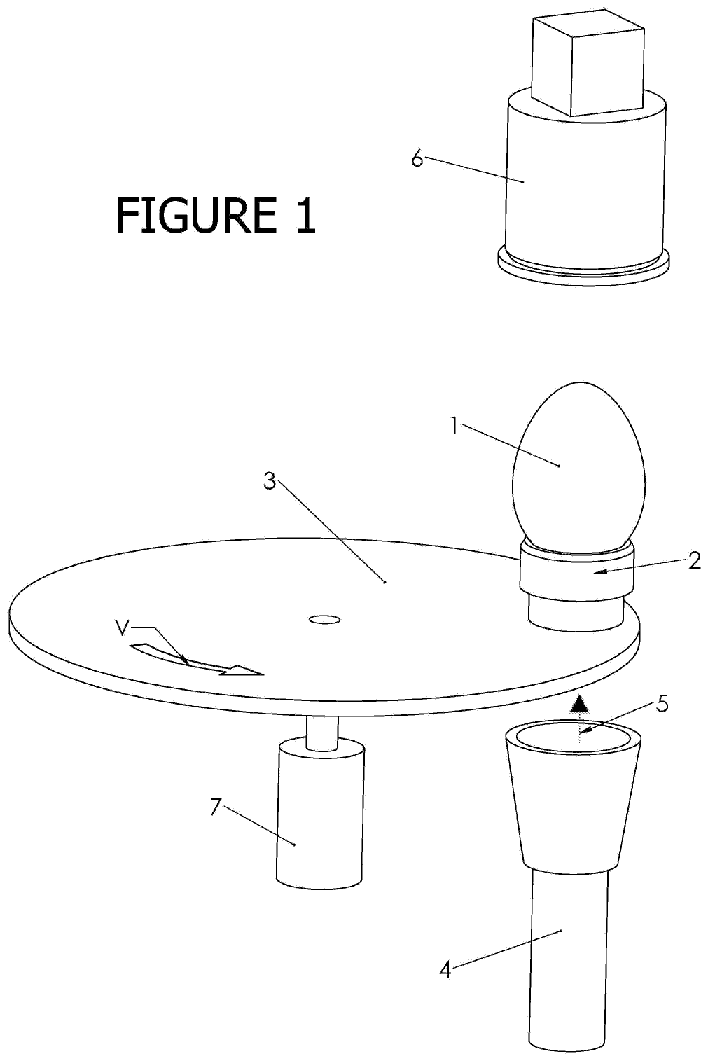

FIG. 1 shows a possible way to achieve the proposed method.

An egg (1) is placed in a hole (2) in the disk (3).

A flashlight (4) located under the hole in the disk illuminates (5) the egg at the bottom through the hole in the disk.

A camera (6) is permanently installed and captures the surface of the eggshell.

The first image or photograph is taken when the disk is at rest in front of the camera.

A motor (7) rotates the disk at a speed that exerts a centrifugal force on the egg, causing the liquid content of the egg to be displaced away from the center of rotation.

When the egg passes exactly where the first image was taken, the camera takes a second image. An additional image can also be taken when the disk rotates at a different speed, which changes the centrifugal force and consequently the position of the liquid content of the egg relative to the eggshell.

The subtraction of all the photographed points of the first image from the second will “eliminate” all the defects and unevenness of the surface of the eggshell and will also reduce the effect of its color.

Having subtracted the second image from the first, what remains will emphasize only the differences in radiation distribution through the liquid content of the egg which changed its place between the two images. From these differences one can deduce the color of the yolk, non-uniformity in the liquid content of the egg, the presence of an embryo or defects in the liquid content of the egg, etc.

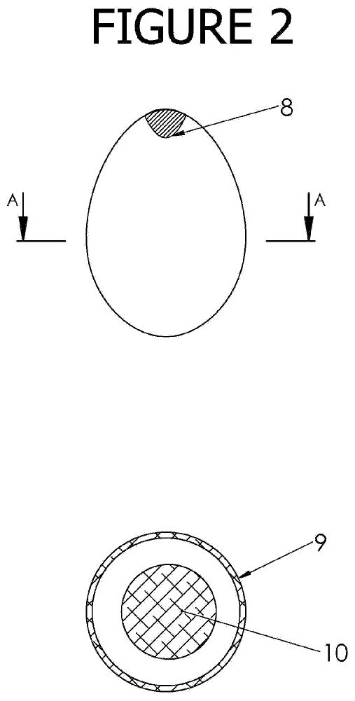

FIG. 2 shows the egg at rest. The distribution of radiation (8) on the eggshell is caused due to the position of the yolk (10) in relation to the eggshell (9). The camera captures the radiation distribution (8).

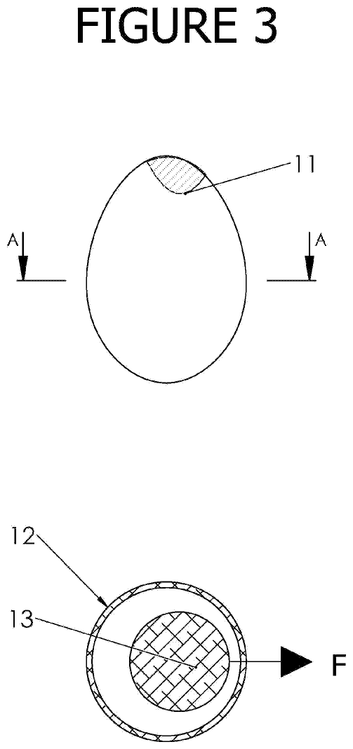

FIG. 3 shows the egg as the disk rotates at speed V1 and a centrifugal force F is applied to the egg. The distribution of radiation (11) across the eggshell changes its position, due to the change in the position of the yolk (13) in relation to the eggshell (12).

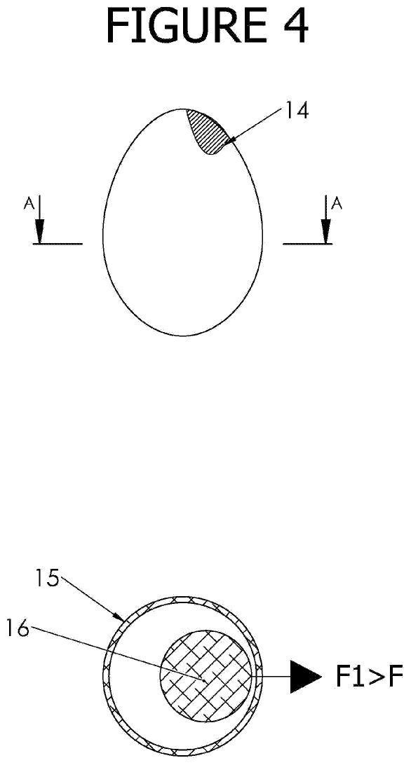

FIG. 4 shows the egg as the disk rotates at speed V2 and a centrifugal force F1 is applied to the egg. The distribution of radiation (14) across the eggshell changes its position, due to the change in the position of the yolk (16) in relation to the eggshell (15).

In cases where a second image is taken close to where the first image of the egg was taken, the eggshell is not positioned accurately in relation to the camera, in which case an adjustment can be made between the two images (using computerized image processing or any other known technique), in a way that makes it possible to distinguish the difference between the images that originated from the deviation of the liquid content of the egg.

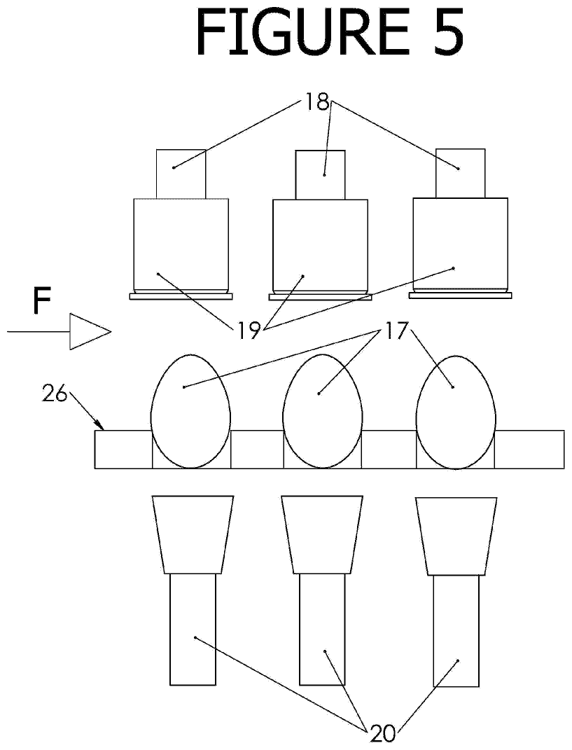

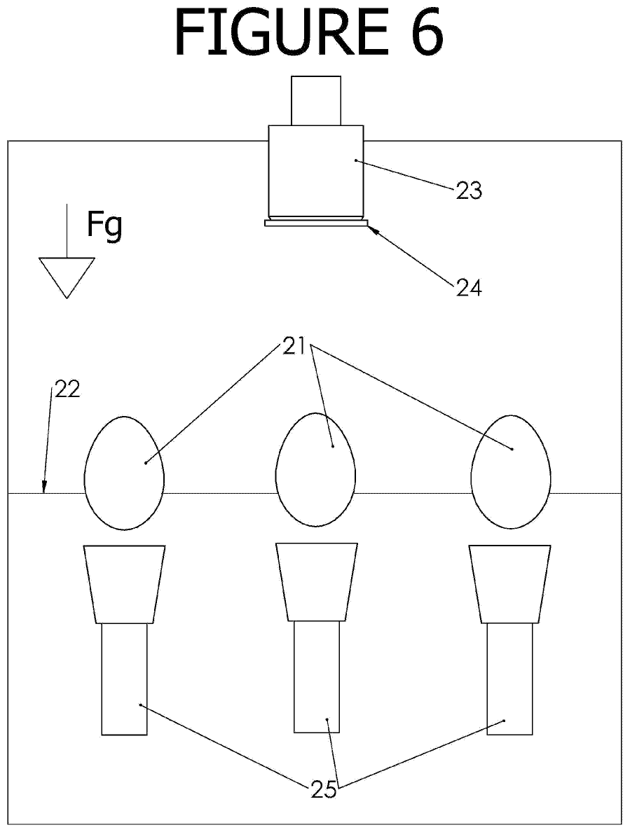

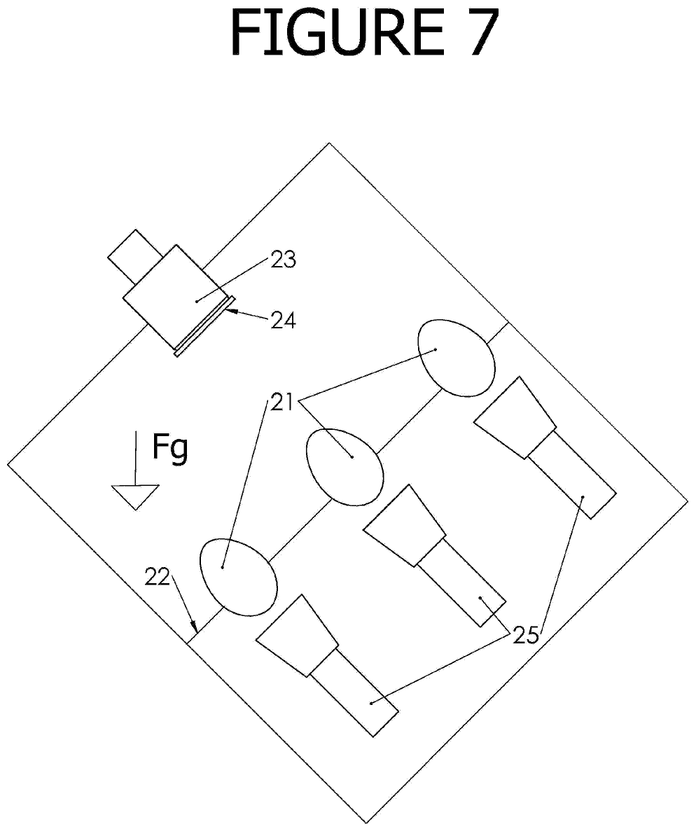

FIG. 5 shows another method of applying a change to force F on several eggs arranged on a moving device (26), such as a conveyor belt or other type of moving surface. The change in force is done by accelerating or decelerating the movement of the moving device. The eggs (17) are placed on the moving device and driven by it. The cameras (18) image the eggs while changing the force exerted on them and the liquid content of the eggs can be analyzed by comparing the images. The cameras can take pictures with or without optical filters (19) that allow passage of different wavelengths. The eggs can be illuminated by an array of light sources (20) continuously or by light flashes, in order to enhance the photographic capabilities.

FIG. 6 shows another method of applying a variable force to an egg or several eggs (21) fixed to a rigid device (22). A camera (23), with or without an optical filter (24), is fixed to the same installation. The array of light sources (25) is also fixed to the facility to allow for improved photography. The diagram shows the first shooting mode, with the device positioned so that the force of gravity (F g), acts perpendicular to all the eggs.

In FIG. 7 the whole device described in the previous method (FIG. 6) and all its fixtures are tilted at an angle so that the direction of the force of gravity F g acting on the eggs changes. Photographing the eggs in this condition and comparing it to the image taken in the previous step (FIG. 6), enables an examination of the liquid content of the eggs.

1. An optical method of examining the liquid content of an egg by illuminating the egg, which results in illumination intensity distribution to appear on the eggshell of the egg, the illumination intensity distribution is dependent on both the structure of the eggshell and the nature of the egg's contents, the method comprising:
a. obtaining at various intervals of time images of the eggshell surface with illumination intensity distribution, wherein at each time interval a force acting on the egg is changed in such a way that the force change does not have any observable effect on the eggshell, but causes a change in the liquid contents of the egg, either in the position of the contents as a whole or in the position of particles within the liquid contents, thus changing the illumination intensity distribution appearing on the eggshell;
b. comparing the obtained images of the eggshell and identifying differences in illumination intensity distributions, thus allowing optical examination of egg's liquid content with minimal optical interference from illumination intensity distribution of the eggshell.

2. The method of claim 1, in which the position of the illuminated egg in one image is not identical to the position of the same illuminated egg in another image, and wherein the positional discrepancy between the images is such that it can be minimized or totally removed by manipulating the images.

3. The method of claim 1, wherein the images are taken by several cameras.

4. The method of claim 1, wherein the images are taken using at least one optical filter.

5. The method of claim 1, wherein the contents of the egg are illuminated with flashes of light.

6. The method of claim 1, wherein the images of several eggs are taken simultaneously.

7. The method of claim 2, wherein the images are taken by several cameras.

8. The method of claim 2, wherein the images are taken using at least one optical filter.

9. The method of claim 2, wherein the contents of the egg are illuminated with flashes of light.

10. The method of claim 2, wherein the images of several eggs are taken simultaneously.

11. The method of claim 3, wherein the images are taken using at least one optical filter.

12. The method of claim 3, wherein the contents of the egg are illuminated with flashes of light.

13. The method of claim 3, wherein the images of several eggs are taken simultaneously.

14. The method of claim 7, wherein the images are taken using at least one optical filter.

15. The method of claim 7, wherein the contents of the egg are illuminated with flashes of light.

16. The method of claim 7, wherein the images of several eggs are taken simultaneously.
